(19) United States
(12) Patent Application Publication
(10) Pub. No.: US 2023/0055893 A1
(43) Pub. Date: Feb. 23, 2023

(54) Hair Oil

(71) Applicant: Shanetta Blackman, Lawrenceville, GA (US)

(72) Inventor: Shanetta Blackman, Lawrenceville, GA (US)

(21) Appl. No.: 17/409,336

(22) Filed: Aug. 23, 2021

(51) Int. Cl. A61K 8/92; A61Q 7/00; A61Q 5/12; A61Q 5/00

(52) U.S. Cl. CPC A61K 8/922; A61Q 7/00; A61Q 5/12; A61Q 5/006

(57) ABSTRACT

An improved composition of hair oil for stimulation of hair growth, control of hair fall, dandruff and infections thereof. The composition of hair oil comprising active ingredients of variety of herbs, vitamins, essential oils and Organic Jojoba. The composition of hair oil supports the hair growth and relaxation of the scalp. The composition of hair oil also effectively reduces/remove dandruff and thereby control hair fall in the subject (e.g., not limiting to male/female human). The composition of hair oil additionally demonstrates the properties of cooling effect, darkening of hair and promotion of hair upon continuous use of the hair oil by the subjects.

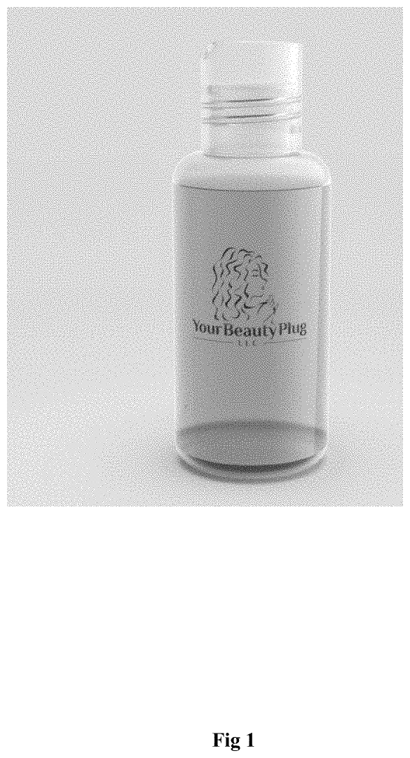

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The invention relates generally to a rejuvenating hair oil system and a method of using the rejuvenating hair oil system. More particularly, the invention relates to a rejuvenating hair oil system which is designed to treat dry and itchy hair, dandruff, cracked scalp, hair loss, and mild rashes resulting in thick and smooth hair.

### Description of the Related Art

In general, the hair in humans is generated by the hair follicles implanted in the scalp. A healthy head of hair is said to contain between 100000 and 150000 hairs, and each hair within this head of hair possesses its own cycle. The life cycle of the hair can be described in three successive physiological phases such as, Anagen, Catagen and Telogen. Anagen is a phase of hair growth which may last from a few weeks to 10 years, Catagen phase is a transient phase of involution of the follicle and ceasing of hair growth with degeneration of the root which may last for a few weeks and Telogen is a phase of shedding of the hair with the root moving up towards the surface by lasting 1 to 5 months. At the end of Telogen phase, the hair therefore disappears from the scalp and this disappearance may extend from a few days to a few months before the follicle is reactivated to give a new hair in the Anagen phase.

Hair thinning is a common problem for both men and women, and can occur as a result of aging, over-treatment with dyes, perms and straighteners or as a common side effect of chemotherapy. Dry or brittle hair breaks off close to the scalp or excessive amounts of hair fallout from the follicle, creating hair loss. People with this type of hair loss want to prevent additional loss by keeping the remaining hair supple and soft and as well as encourage new growth from the follicles in the scalp. Many products for the hair have ingredients that can contribute to hair loss, especially if the hair is fragile or previously damaged. Ninety percent of shampoos contain surfactants such as sodium lauryl sulfate, a harsh surfactant that is irritating to the scalp and strips the hair, making it weaker and more prone to becoming damaged.

Hair thinning and loss is often a cause of great psychological stress to the person symbolizing a loss of youth, health and vitality. Many people try to coax the hair into regrowing by various treatments applied topically to the scalp or by ingesting certain drugs. Most experience unsatisfactory results. For example, the FDA has approved two drugs for the treatment of male pattern baldness, but only a lower strength of one of the drugs is approved for use by women with alopecia or baldness. Neither drugs address the quality of the hair that may grow back. Other vitamins, minerals and botanical extracts have been proposed for oral ingestion to improve the quality and quantity of the patient's hair.

Dandruff is a common affliction most usually associated with the human scalp area. It is recognized that skin normally sloughs off the skin surfaces of the human body. In general practice, the skin which is sloughed off is normally washed away at frequent intervals so that the sloughing off process is not noticeable. On the scalp, which is not normally washed as often as other parts of the body, the sloughed-off skin tends to accumulate. This skin together with the natural oils exuded by the scalp form a suitable environment for the growth of bacteria, and in fact certain bacteria are known to be associated with hair fall or hair loss.

Many cosmetic products have been tried over the centuries from bird droppings in ancient times to today's various amino acids, vitamins, minerals, colloidal silver, copper chelates and botanical extracts such as saw palmetto, and tree nut oils, all claiming to promote hair regrowth, to improve the appearance of the hair, making it thicker and more luxurious. Most are applied to the hair, and the scalp, and one has been proposed for application into the scalp.

There are multiple solutions that have been presented in prior art. However, these solutions are limited and restricted to their conventional architecture, installation system and have considerable shortcomings which adversely affect the convenience with which they can be used. The prior systems have certain limitations including the ingredients and effects which make them not suitable for every type of user. Moreover, the ease-of-use ability of these assemblies is also questionable.

Therefore, it would be advantageous to design an improved composition of hair oil for stimulation of hair growth, control of hair fall, dandruff and infections thereof, as described in greater detail herein.

None of the previous inventions and patents, taken either singly or in combination, are seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of a hair oil for a dry and itchy scalp or hair, dandruff, cracked scalp, hair loss, and mild rashes.

It is also the objective of invention to provide Hair Growth Oil and Mask with natural and organic ingredients for thick and smooth hair.

Another object of the present invention is to provide oil which is formulated with Argan Oil and Coconut Oil to revive hair and skin,

Another object of this invention is to provide a new and improved combination of hair oil where oats are included in the formula containing Organic Jojoba as well as exfoliation properties that unclog pores and facilitates while promoting hair growth.

It is an object of the invention in one of its aspects to provide a combination which, stimulate the blood vessels when massaged through the scalp and skin.

It is a further aspect of the present invention to provide a new and improved hair oil which is developed from reliable materials and sustains its effectiveness over a longer period of time.

Thus, it is the objective to provide a new and improved assembly of an improved hair growth product for stimulation of hair growth, control of hair fall, dandruff and infections thereof. Other aspects, advantages and novel features of the present invention will become apparent from the detailed description of the invention whet considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved hair growth product for stimulation of hair growth, control of hair fall, dandruff and infections thereof.

The aforementioned aspects and other objectives and advantages can now be achieved as described herein. An improved composition of hair oil for stimulation of hair growth, control of hair fall, dandruff and infections thereof. The composition of hair oil comprising active ingredients of Organic Argan Oil, Coconut Oil, Organic Jojoba, and Shea Butter.

The composition of hair oil supports the hair growth and relaxation of the scalp. The composition of hair oil also effectively reduces/removes dandruff and thereby control hair fall in the subject (e.g., not limiting to male/female human). The composition of hair oil additionally demonstrates the properties of cooling effect, darkening of hair and promotion of hair upon continuous use of the hair oil by the subjects. In an alternative embodiment of the present invention, the proposed composition of oil is alternatively used to reduce/remove Psoriasis.

The clinical trial study results prove the efficacy of the proposed composition of hair oil, the composition of hair oil has greater efficacy in controlling the dandruff and thereby promoting improved hair growth in the subjects.

The process of creating hair oil starts with collecting all of the natural and organic herbs and measuring each herb individually to meet the required batch amount based on how many bottles that needed for production. All herbs are kept at room temperature and are precisely measured and separated before the bottling process begins. Once each bottle is prepped with herbs, the process of making the oil begins by infusing all of the required ingredients based upon batch amount needed for production. After all oils are infused the bottling process begins by filling each bottle with the measured amount for each bottle size. The last step in the production process is storing all bottles in a cool area at room temperature for 14 days, finishing the infusion process for the selling. All bottles are separated and labeled by production dates to assure that all the ingredients combined act together as one to assure premium results.

The effect of the composition of hair oil on hair population, in accordance with the disclosed embodiments. The results reveal that there is good increase in the hair population of the subjects on continuous use of the test oil over the study period. Before study start clinicians have graded the hair population of 90% of the subjects under ‘fair’ category and remaining 10% between ‘poor and fair’ category while at the end of the study period clinicians had rated the hair population of the subjects in the grades of ‘fair-good-good-good-excellent’. It should be noticed that the clinicians have indicated appreciable changes in the subjects' hair population after 60 days of oil use.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A method for stimulation of hair growth, control of hair fall, dandruff and infections consisting essentially of administering a composition consisting essentially of therapeutically effective amounts of:
Oils including Organic Argan Oil, Coconut Oil, Organic Jojoba, and Shea Butter

2. A hair oil which can be used daily to enjoy the benefits of stronger, thicker, luscious hair. The hair oil provides essential nutrients to promote and stimulate natural hair growth.
